(19) United States
(12) Patent Application Publication
(10) Pub. No.: US 2003/0158546 A1
(43) Pub. Date: Aug. 21, 2003

(54) HIGH FREQUENCY SURGICAL INSTRUMENT

(76) Inventors: GUNTER FARIN, TUBINGEN (DE); PETER PUTZ, TUBINGEN (DE); REINER HAAG, PFAFFINGEN (DE)

Correspondence Address: MICHAEL E. ATTAYA, CESARI AND MCKENNA, LLP, 88 BLACK FALCON AVENUE, BOSTON, MA, 02210, US

(21) Appl. No.: 08/764,037

(22) Filed: Dec. 12, 1996

Related U.S. Application Data
(63) Continuation of application No. 08/619,861, filed on Mar. 19, 1996, now abandoned.
(63) Continuation of application No. 08/433,911, filed on May 30, 1995, now abandoned.
Application No. 08/433,911 is a continuation of application No. 08/048,545, filed on Apr. 16, 1993, now abandoned.
Application No. 08/048,545 is a continuation of application No. 07/914,491, filed on Jul. 7, 1992, now abandoned.
Application No. 07/914,491 is a continuation of application No. 07/715,845, filed on Jun. 14, 1991, now abandoned.
Application No. 07/715,845 is a continuation of application No. 07/585,042, filed on Sep. 19, 1990, now abandoned.
Application No. 07/585,042 is a continuation of application No. 07/467,275, filed on Jan. 23, 1990, now abandoned.
Application No. 07/467,275 is a continuation of application No. 07/355,120, filed on May 19, 1989, now abandoned.
Application No. 07/355,120 is a continuation of application No. 07/244,537, filed on Sep. 12, 1988, now abandoned.
Application No. 07/244,537 is a continuation of application No. 06/921,540, filed on Oct. 21, 1986, now abandoned.

(51) Int. Cl. A61B 18/12

(52) U.S. Cl. 606/34; 606/38; 606/39; 606/40

(57) ABSTRACT

Described is a high-frequency surgical instrument with automatic control of the intensity of the electric arcs between the active electrode and the tissue that is to be cut or coagulated, whereby for determination of the extent of the electric arcs, a therefrom dependent signal (K) is shunted off through a filter and conducted to a rectifier whose output signal is conducted to a variable-gain amplifier in which this output signal is compared either with the set-point signal (S) of a set-point device for the cutting process or with the set-point signal (K) of a set-point device for the coagulation process. The variable-gain amplifier generates a signal proportional to the difference of the signals E minus S or E minus K, respectively, which is conducted to an amplitude modulator which controls the amplitude of the output voltage of the power amplifier. The filter is designed so that it filters-out at least one frequency of the nonharmonic frequencies of the basic frequency of the high-frequency oscillator, generated by the electric arcs between active electrode and tissue, from the electric voltage or from the electric current in the output and simultaneously strongly attenuates the basic frequency and its harmonic frequencies so that they have no effect on the control signal.

### BACKGROUND OF THE INVENTION

This invention relates to a high-frequency surgical instrument for the cutting and/or coagulating of biologic tissue with automatic monitoring or control of the high-frequency current during the cutting and/or during the coagulation, whereby there is used as a criterion for the control or monitoring the intensity of the electric arcs between the active electrode and the tissue which is to be cut or coagulated.

The invention takes as a point of departure the recognition that the high density of the electric current required for the cutting of biologic tissues can, as a rule, be attained only when the so-called active electrode does not touch the tissue directly but when there arises, given sufficiently high electric voltage between the tissue and the active electrode, an electric arc which concentrates the total electric current point-like onto the site of the tissue that is to be cut.

This recognition is not new, but has been ignored. In the year 1909, there was achieved by DE FOREST for the first time a cutting effect by means of an electric spark jumping from an electrode onto the tissue. The so-called spark-cut according to DE FOREST was introduced in Germany from 1910 on by M. COHN and von CZERNY. The so-called spark-cut was further described in detail by J. KOWARSCHIK (1928) and H. V. SEEMEN (1932), both by Verlag J. Springer, Berlin. More recent authors, on the other hand, describe as cause for the cutting effect the high current density of the electric current which is achieved on small-area direct contact between the active electrode and the tissue-to-be-cut without reference to an electric arc or spark. In German Patent DE 31 19 735 AL is described a “Method and a Circuit Arrangement for Controlling the Output Power of an Electrosurgical High-Frequency Generator” by means of which “the discharge current density at the cutting electrode remains limited to a lower value than is necessary for the ignition of an electric arc”.

In the German patent publication DAS 28 01 833 B1, the inventor takes as point of departure the hypothesis that the cutting effect depends on the temperature of the cutting electrode so that it is the object of his invention to keep the temperature of the cutting electrode during the cutting in a desired temperature range. The inventor mentions a D.C. voltage that develops proportional to the temperature of the cutting electrode between the cutting electrode and the tissue, where the object is to regulate the temperature so that the temperature of the cutting electrode can be kept in the desired temperature range.

Both in DE 31 19 735 A1 and DAS 28 01 833 B1, the causal significance of the electric arc in relation to the cutting effect of the electric current is not properly recognized so that the proposed methods for the regulation of the output power of the high-frequency generators for high-frequency surgical cuts are based on incorrect hypotheses.

DP 25 04 280 describes a device for the cutting and/or coagulation of human tissue with high-frequency current which takes as point of departure the causal importance of the electric arc between the active electrode, there called a probe, and the tissue that is to be cut or coagulated. This instrument is characterized by an indicating device which indicates the intensity of an electric arc between the probe and the tissue by means of an electric signal. The electric signal of the indicating device and a set-point program are provided to a regulating device and the regulating device derives from these two data a control quantity and feeds it to the generator in such a way that the current intensity of the high-frequency current is always set at a value that corresponds to the set-point program and the desired extent of the electric arc.

In order to acquire an electric signal suitable for the control, it is proposed in DP 25 04 280 to analyze the starting point of the operating range and the strength of the electric arc by measuring the strength of the currents of the harmonic frequencies of the operating frequency conducted to the probe.

As high-frequency power generators generate at the highest possible degree of efficiency as a rule, they generally produce harmonic frequencies of the basic frequency, the level of which increases with increasing efficiency. DP 25 04 280 proposes that the indication device contain a filter between the generator and the probe, which conducts currents of the operating (basic) frequency and which blocks currents of the harmonic frequencies. The measuring of the currents of the harmonic frequencies takes place at the outlet of the filter. Because of the insertion of a lowpass filter into the output circuit of the high-frequency generator, one must expect a lower degree of efficiency.

On the practical realization of a high-frequency surgical instrument corresponding to DP 25 04 280, it has been found that the filter expenditure for suppression of the currents of the harmonic frequencies of conventional high-frequency generators is relatively high and that the degree of efficiency becomes relatively low.

In order to realize the two demands on the high-frequency generator for the device for the cutting and/or coagulation in accordance with DP 25 04 280, that is, having the smallest possible participation of currents of harmonic frequencies of the basic frequency and the highest possible degree of efficiency, on the other hand, there is used in the serially manufactured high-frequency surgical instrument according to DP 25 04 280 a special, selective amplifier according to DP 32 27 109.3. This selective amplifier contains, however, rather expensive selective resonance circuits and filters which moreover must be adjusted relatively accurately to the basic frequency or its harmonic frequencies, respectively.

It is therefore an object of the invention to develop a high-frequency surgical instrument with automatic control of the intensity of the electric arcs between the active electrode and the tissue which is to be cut or coagulated which, compared to the known high-frequency surgical instrument of DP 25 04 280, requires less expenditure on filtering and filter balancing and which can also be operated without a special, selective power amplifier.

Extensive investigations about the processes at the active electrode during the cutting and/or coagulation resulted in the recognition that the electric arc required for the cutting of biological tissues is highly variable both in time and place between the active electrode and the tissue. As soon as the electric field intensity is sufficiently high at one or more sites between the active electrode and the tissue, there are ignited at this point or points several electric arcs. At the point at which the specific electric arc touches the biologic tissue, the tissue is so strongly heated that the water content and other volatile constituents of the tissue vaporize immediately. Thus, the respective tissue part immediately disappears after contact with the electric arc and there arises in its place a partial crater. At the location of the partial crater, the distance between the active electrode and the biologic tissue increases and the electric field intensity becomes correspondingly lower just at this point so that the electric arc either disappears at this partial site, softens into the edge of the created crater or ignites at another partial site between the active electrode and the tissue, that is, where there exists the next favorable condition for ignition. As soon as an electric arc has ignited at any particular partial site between the active electrode and the biological tissue, it undermines within a short time the basis of its existence at the site at which it ignited so that it has to swerve to other sites or must newly ignite at other points. This process proceeds stochastically and at very high speed. The sum total of all thus created partial craters forms the cut or the cutting fissure in the biological tissue.

As a result of these electric arcs stochastically and with high speed igniting and extinguishing, the span between the active electrode and biologic tissue has the behavior of an electric resistance with strong, broadband noise. Taking it as a model, this span can also be considered as a noise source whose noise output is proportional to the intensity of the electric arcs. The proportionality between noise output and intensity of the electric arcs is sufficiently good to use it as a criterion for control of the intensity of the electric arcs.

The electric arc between the active electrode and the tissue, as is long known, has a nonlinear proportionality (resistance) between current and voltage, which provides a rectification effect and extends the frequency spectrum of the noise from the frequency zero until far above the basic frequency of the high-frequency generator.

When using a selective frequency or a more or less broad frequency band from the very broad frequency spectrum of the noise, it is advantageous with a view to a highest possible control rate to use the highest possible frequencies from this spectrum. However, in consideration of the decline of the amplitude, only frequencies below f₆ are of practical interest. The harmonic frequency f₀ (equal to 0 Hz) is not suitable for control because the acting time constant would be infinite.

In any case, however, it has to be kept in mind that the basic frequency and the harmonic frequencies of the basic frequency which are supplied by the high-frequency generator of the high-frequency surgical instrument are not suitable for the control. All other frequencies of the noise caused by the electric arc are suitable to a greater or lesser degree.

In order to obtain an electric signal that is sufficiently proportional to the intensity of one of the several electric arcs between the active electrode and the tissue, an electric filter is needed which filters-out from the noise caused by the electric arc either a single frequency other than the basic frequency or harmonic frequencies of the high-frequency generator, or several frequencies of more or less broad frequency bands, exclusive of the basic frequency and the harmonic frequencies of the high-frequency generator.

The nonharmonic frequencies can be filtered-out in principle either from the electric voltage between the active electrode and tissue or from the electric current which flows through the active electrode into the tissue. Care must be taken that the intensity of low-frequency currents through the tissue remains below the electric stimulus threshold of nerve and muscle cells.

This solution is particularly advantageous in comparison to the known high-frequency surgical instrument of DP 25 04 280 in as far as the output amplifier of the high-frequency surgical instrument of the invention does not have to be either selective or free of harmonic frequencies of the basic frequency. Nor does it have to be especially frequency-stable. This solution is also advantageous in as far as there is only required a filter to obtain a signal suitable for the control; the filter does not have to transmit any large power and can therefore be manufactured relatively small in size and cost-effectively.

While there are required, as described above, electric arcs between the active electrode and the tissue for the cutting, such arcs have to be avoided as much as possible during the coagulation.

For cutting processes, set-point devices are required which control the amplitude of the output of the power amplifier of the high-frequency surgical instrument at a sufficiently high level that electric arcs are constantly present between the active electrode and the tissue.

On the other hand, for coagulation processes, set-point devices are required which control the amplitude of the output of the power amplifier of the high-frequency surgical instrument so that no electric arcs can arise between the active electrode and the tissue or that so few electric arcs exist that no cut is performed.

For coagulating-cutting processes where the cut surfaces during the cutting process must simultaneously be more or less strongly coagulated, there are required set-point devices which control in sufficiently quick sequence the amplitude of the output signal of the power amplifier of the high-frequency surgical instrument in such a way that intervals of coagulation and cutting succeed one another. Such set-point devices are known from DP 35 30 335.2.

### SUMMARY OF THE INVENTION

This application describes a high-frequency surgical instrument with automatic control of the intensity of the electric arcs between the active electrode and the tissue that is to be cut or coagulated, whereby for determination of the intensity of the electric arcs, a therefrom dependent signal (E) is shunted off through a filter and conducted to a rectifier whose output signal is conducted to an amplifier in which this output signal is compared either with the set-point signal (S) of a set-point device for the cutting process or with the set-point signal (K) of a set-point device for the coagulation process. The amplifier generates a signal proportional to the difference of the signals E minus s or E minus K, respectively, which is conducted to an amplitude modulator which controls the amplitude of the output voltage of the power amplifier. The filter is designed so that it passes at least one frequency of the nonharmonic frequencies of the basic frequency of the high-frequency oscillator, generated by the electric arcs between active electrode and tissue, either from the electric voltage or from the electric current in the output, and simultaneously strongly attenuates the basic frequency and its harmonic frequencies so that they have no effect on the control signal.

### BRIEF DESCRIPTION OF THE DRAWINGS

The invention is described hereunder in more detail with the aid of the drawings FIG. 1 to FIG. 5.

FIG. 1 is a schematic presentation of the frequency spectrum of a high-frequency generator of a customary known high-frequency surgical instrument while no electric arc is present between the active electrode and the tissue.

FIG. 2 is a schematic presentation of the frequency spectrum of the current or voltage between the active electrode and the tissue while electric arcs are present.

FIG. 3 is a schematic presentation of the frequency spectrum of the current or voltage between the active electrode and the tissue while electric arcs are present.

FIG. 4 illustrates the criteria for the configuration of a high-frequency filter.

FIG. 5 is an exemplified embodiment of a high-frequency surgical instrument which incorporates the present invention.

### DETAILED DESCRIPTION OF THE PREFERRED EMBODIMENT

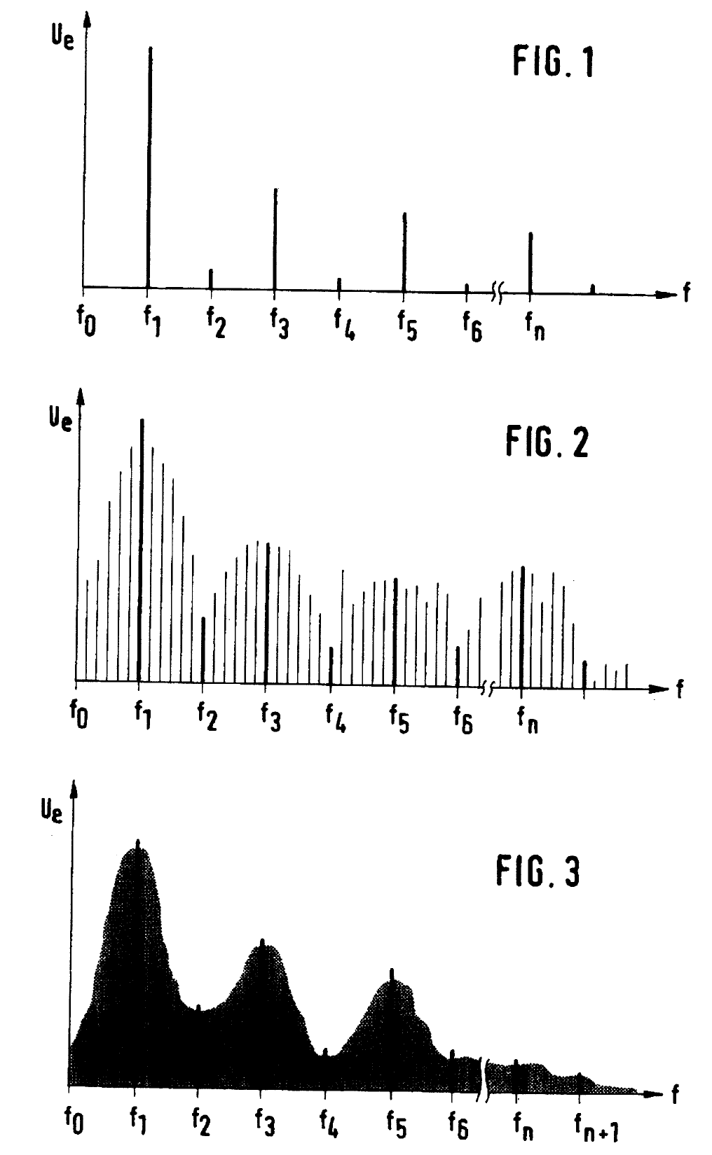

FIG. 1 shows a typical frequency spectrum of a known high-frequency generator for high-frequency surgery. As these high-frequency generators are used to obtain a high degree of efficiency with a view to the dissipation loss within the high-frequency generator with push-pull-amplifiers in B- or C- or even D-operation, these generators furnish, besides the basic frequency f₁, more or less intense integral multiples of the basic frequency f₁, the so-called harmonic frequencies f₂, f₃, f₄, f₅, f₆ . . . fₙ. The odd harmonics f₃, f₅, etc. are present, as a rule, more intensively than the even harmonics f₂, f₄, etc.

As long as no arc is present between the active electrode and the tissue, there is present at the outlet of the high-frequency generator only the basic frequency f₁, as well as more or less intensely the integral harmonic frequencies of the basic frequency. The zero-harmonic frequency f₀ is not present.

As soon as electric arcs are, however, present between the active electrode and the tissue, there arises, as a result of the nonlinear behavior of the electric resistance of the electric arcs and the stochastic abundance of the electric arcs per unit of time, nonharmonic frequencies between the harmonic frequencies as is represented schematically in FIG. 2. As the nonharmonic frequencies caused by the electric arcs appear stochastically, their statistical distribution is without gaps, as schematically presented in FIG. 3.

As a criterion for the automatic control of the intensity of the electric arc between the active electrode and the tissue, there is selectively filtered out from the total spectrum represented in FIG. 2 or FIG. 3 and prepared as a control signal a defined nonharmonic frequency of a more or less broad frequency band between two adjacent harmonic frequencies or several more or less broad frequency band of the nonharmonic frequencies between different adjacent harmonic frequencies.

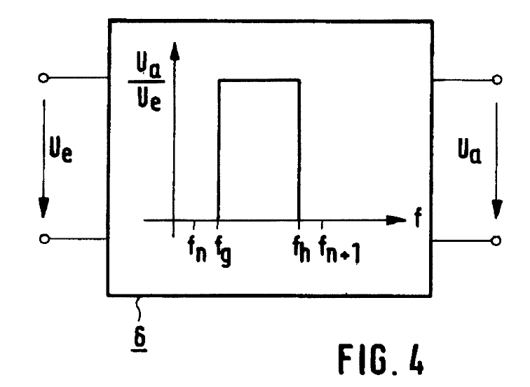

FIG. 4 shows the criteria that have to be taken into account in the automatic control of the intensity of the electric arcs between the active electrode and tissue. The harmonic frequencies fₙ and fₙ₊₁, whereby n can be any integral number from zero onwards, are not suitable for the automatic control when the high-frequency generator of the respective high-frequency surgical instrument does not supply these frequencies with non-negligible level. Because the basic frequency f₁ of the high-frequency generators of high-frequency surgical instruments is not fixed accurately to a standardized norm in view of the expense, the basic frequency used in each case is not very stable. Also, because of amplitude modulation through mains hum and automatic control there arise side frequency bands. For the generation of the automatic control signal, only such nonharmonic frequencies, out of the respective frequency interval fₙ to fₙ₊₁, can be used which have sufficient distance from the harmonic frequencies inclusive of their tolerances and side frequencies. In FIG. 4, there are schematically presented two safety margins f_{g}-fₙ and fₙ₊₁-fₕ. On the selection of the bandwidth fₕ-f_{g}, it is beneficial, in consideration of the stochastic probability of the nonharmonic frequencies generated through the electric arcs, to select them as broadly as practically realizable, whereby the rate of rise of the forward function Uₐ/Uₒ of the bandpass filter will be as large as possible.

The forward function Uₐ/Uₒ presented in FIG. 4 over f is idealized. On the selection and dimensioning of this filter, an attempt should be made to approach this idealized forward function as closely as possible. There are known many different high-frequency filter circuits, for example, piezoceramic filters (e.g. Cat. No. p4E-3, 1984 of the firm MURATA Mfg. Co., Ltd.), that meet this requirement.

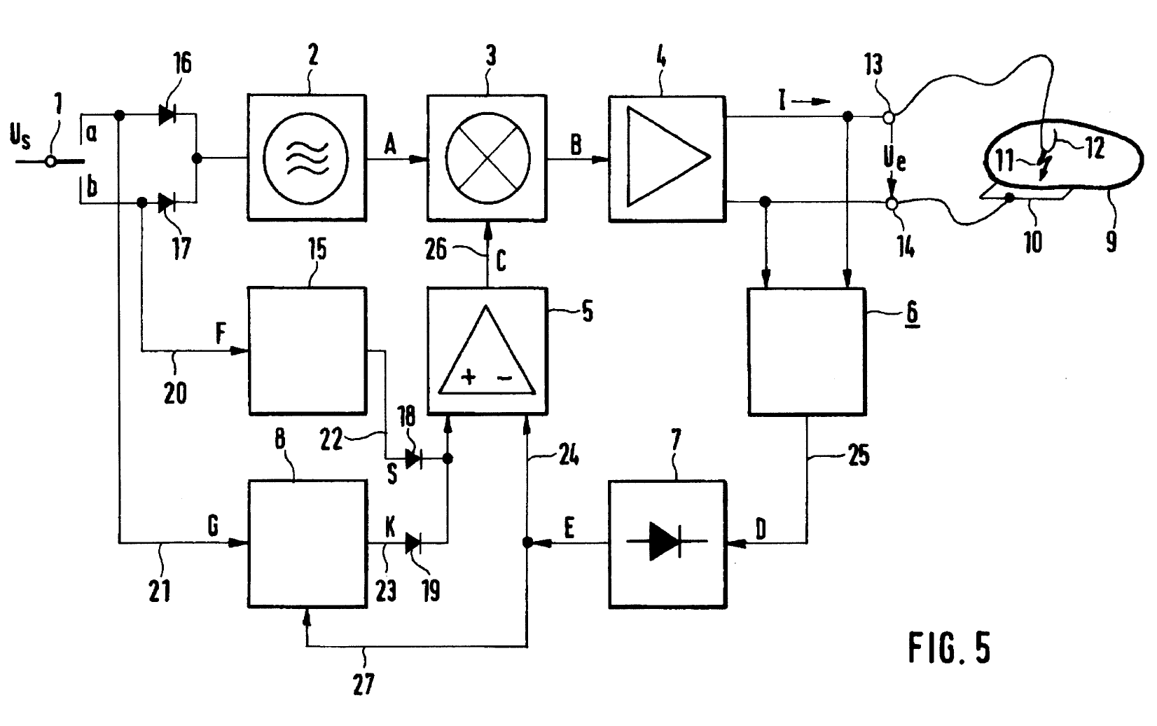

FIG. 5 shows the invention-relevant details of a high-frequency surgical instrument of the invention consisting of a high-frequency oscillator 2, an amplitude modulator 3, a power amplifier 5, a filter 6 for the nonharmonic frequencies as discussed above with respect to FIG. 4, a rectifier 7, a set-point device 8 for coagulation, a set-point device 15 for cutting, and a test switch 1 for the activation of the high-frequency generator either for coagulation when contact a is closed, or for cutting when contact b is closed.

At outlets 13, 14 of the high-frequency surgical instrument are connected an active electrode 12 and a neutral electrode 10. The neutral electrode 10 is conductively applied to a tissue 9 of the patient.

When the test switch 1 is switched in switch position a, there is activated through a diode 16 the high-frequency oscillator 2 and simultaneously there is switched on through a line 21 the set-point device 8 for the intensity of the coagulation power. A set-point signal K for the coagulation power is conducted through a line 23 and a diode 19 to the variable-gain amplifier 5. As long as no electric arc ignites between the active electrode 12 and the tissue 9, which are in contact with each other, the output power of the output amplifier 4 is steered by the set-point device 8. As soon as electric arcs ignite between the active electrode 12 and the tissue 9 and thereby create nonharmonic frequencies in the high-frequency output circuit 13, 14, the nonharmonic frequencies suitable for control of the intensity of the electric arcs 11 are passed by the filter 6, are conducted through a line 25 to the rectifier 7, there rectified and then conducted through a line 24 to the amplifier 5.

Because the output signal E of the rectifier 7 is highly proportional to the intensity of the arcs 11, the output power of the output amplifier.4 is so controlled through an output signal C of the amplifier 5, by means of the amplitude modulator 3, such that the intensity of the electric arcs 11 is regulated to the set-point value of the set-point device 8.

The output signal E of the rectifier 7 is conducted through a line 27 simultaneously to the set-point device 8 so that the set-point values for coagulations can be dynamically conducted such that the output power of the power amplifier, after the igniting of the electric arcs 11, is more or less rapidly lowered so that the electric arcs 11 are extinguished, whereupon the output power of the output amplifier 4 is more or less rapidly again regulated upwards until electric arcs 11 again ignite. These cycles repeat themselves as long as the test switch 1 remains in switch position a. Set-point devices suitable for this are described in the DP 25 04 280 and in the DP 35 30 335.2.

When the test switch 1 is switched to switch position b, there is activated through a diode 17 the high-frequency oscillator 2 and simultaneously there is switched on through a line 20 the set-point device 15 for the intensity of the cutting power. The set-point signal S for the cutting power is conducted through a line 22 and a diode 18 to the variable-gain amplifier 5. As long as no electric arc ignites between the active electrode 12 and the tissue 9, which are in contact with each other, the output power of the power amplifier 4 is steered from the set-point device 15. As soon as electric arcs 11 ignite between the active electrode 12 and the tissue 9 thereby creating nonharmonic frequencies in the high-frequency output-circuit 13, 14, the nonharmonic frequencies suitable for control of the intensity of the electric arcs 11 are passed through the filter 6, conducted through the line 25 to the rectifier 7, there rectified and conducted through the line 24 to the variable-gain amplifier 5. Because the output signal E of the rectifier 7 is highly proportional to the intensity of the electric arcs 11, the output power of the power amplifier 4 is controlled through the output signal of the variable-gain amplifier by means of the amplitude modulator 3 such that the intensity of the electric arcs 11 is regulated to the set-point value of the set-point device 15.

What is claimed as new and desired to be secured by Letters Patent of the United States is:

1. In a high-frequency surgical instruments with automatic control of the intensity of electric arcs between an electrode and a tissue which is to be cut or coagulated by controlling the power applied by a high frequency generator (2, 3, 4) to the electrode, the improvement including means for sensing substantially only components of the output of said generator other than the fundamental frequency of said generator and harmonic thereof and means responsive to said components for controlling the output power of said generator.

2. The high-frequency surgical instrument according to claim 1 wherein said sensing means has a bandwidth (f_{g}-fₕ) which is located between the fundamental frequency of said generator and the first harmonic thereof or between two adjacent harmonics, whereby the width of said bandwidth is selected such that the sensing means is substantially unaffected by fluctuations in the fundamental frequency of said generator or by modulation produced by said surgical instrument.

3. The high-frequency surgical instrument according to claim 1 wherein the sensing means comprises a piezoelectric ceramic filter.

4. The high-frequency surgical instrument according to claim 2 wherein the sensing means comprises a piezoelectric ceramic filter.
